(19) United States
(12) Patent Application Publication
(10) Pub. No.: US 2014/0031677 A1
(43) Pub. Date: Jan. 30, 2014

(54) Apparatus and Method for Aiding Needle Biopsies

(71) Applicant: PHYSICAL SCIENCES, INC., (US)

(72) Inventors: Nicusor V. Iftimia, Chelmsford, MA (US); Mircea Mujat, Acton, MA (US); R. Daniel Ferguson, Melrose, MA (US); Daniel X. Hammer, Silver Springs, MD (US); Shing Chang, Andover, MA (US)

(73) Assignee: PHYSICAL SCIENCES, INC., Andover, MA (US)

(21) Appl. No.: 13/743,632

(22) Filed: Jan. 17, 2013

Related U.S. Application Data
(60) Provisional application No. 61/589,082, filed on Jan. 20, 2012.

(51) Int. Cl. A61B 5/00; A61B 10/02

(52) U.S. Cl. CPC A61B 5/0066; A61B 10/02
USPC 600/425

(57) ABSTRACT

A handheld optical coherence tomography imaging and tissue sampling system and method of imaging and sampling a tissue is disclosed. The method includes inserting a catheter probe into a biopsy needle. The biopsy needle can be attached to a hand-held scanning and sampling device. The biopsy needle is maneuvered to an investigation site. A three-dimensional image of the tissue at the investigation site is captured with the catheter probe.

### RELATED APPLICATIONS

This application claims the benefit of and priority to U.S. Provisional Application No. 61/589,082, filed Jan. 20, 2012, which is owned by the assignee of the instant application and the disclosure of which is hereby incorporated herein by reference in its entirety.

### STATEMENT OF GOVERNMENT INTEREST

The subject matter described herein was developed in connection with funding provided by the National Institute of Health under Grant Nos. 2R44CA117218 and 5R42CA114896. The Federal government may have rights in the technology.

### TECHNICAL FIELD

The invention relates generally to an apparatus and method for aiding needle biopsies, and more particularly, to an optical coherence tomography (“OCT”) imaging and tissue sampling system and method.

### BACKGROUND

Early diagnosis and a proper stage determination are key factors that contribute to cancer cure success. A cancer's stage is based on the primary tumor's size and whether it has spread to other areas of the body. If cancer cells are present only in the layer of cells where they developed and have not spread, the stage is in situ. If cancer cells have penetrated the original layer of tissue, the cancer is invasive. When a cancer-suspect mass is identified with radiological imaging, a percutaneous biopsy (e.g., inserting a needle into the patient to collect a tissue sample) is usually performed to further investigate that mass, determine the stage, and determine the most effective therapy option.

Percutaneous biopsy has become established as a safe, effective procedure for cancer diagnosis. Successful percutaneous needle biopsy has been applied in most organ systems with excellent results and few complications. The key to these procedures has been the use of imaging guidance, which allows for the safe passage of a needle into an organ or mass, to obtain tissue for cytologic or histologic examinations. Image-guided biopsy is less invasive than open exploration to obtain these same tissues. Because of the lower morbidity and mortality of the minimally-invasive procedures, image-guided biopsy can be applied to patients who are too ill to undergo surgery or who wish to avoid convalescence from large diagnostic laparotomy procedures. In most settings percutaneous or transcutaneous biopsy is the first approach to diagnosis.

Biopsy is the best current method for confirming the presence of cancer. Core biopsy is the preferred method of tissue acquisition for obtaining adequate material cancer biomarkers analysis. Unfortunately, the difficulty in sampling enough biological material and heterogeneity of tissue, especially in patients that had surgery of radiotherapy, can cause biopsy sensitivity/specificity to vary within a large range (e.g., about 70% to about 95%). If biopsy results are negative, the clinician is put in a difficult situation because the clinician has to decide how to manage the patient when contradictory results from investigations are obtained. Therefore, any measure to improve biopsy success adequate for biomarker analysis would be considered highly significant.

### SUMMARY OF THE INVENTION

What is needed is instrumentation and an approach for aiding transcutaneous and endoscopic biopsies with the goal of increasing the success rate of biopsies. The approach is based on the use of optical coherence tomography (“OCT”), which is a high-resolution imaging technique that can be used to assess tissue cellularity at the tip of a biopsy needle. OCT has proven its capability to image tissue morphology at the micron scale, and thus has enabled the diagnosis of cancer disease in the early stage, at the epithelial level. However, the use of OCT for imaging interstitial tissue is somewhat limited due to the lack of minimally invasive probes that can be deployed alone, or by means of biopsy needles, to image tissue morphology in various organs, for example, lungs, breast, pancreas, liver or kidney, where a natural lumen does not exist or cannot be used to image deeper within the organ. OCT applicability and translation to clinical use, especially for imaging interstitial tissue, has been limited by the lack of adequate probes that can be deployed safely without significantly disrupting tissue morphology and inducing severe bleeding, while providing high-quality images.

Embodiments of the invention relate to apparatuses and methods for differentiating tissue types using OCT imaging. The apparatuses and methods can aid biopsies performed through a long needle, usually computed tomography scan (“CT Scan”) or ultrasound guided to the biopsy site, or biopsies through a regular short biopsy needle. A hand-held OCT probe and scanning engine can be used to perform tissue sampling through a biopsy needle. For longer biopsy needles, the OCT probe can be controlled by a scanning engine placed close to the proximal end of the needle. A tissue differentiation algorithm can be implemented via software on a processor to differentiate between different types of tissue. This tissue differentiation algorithm can differentiate between various tissue types: adipose (e.g, body fat), fibrous (e.g., connective tissue), cancer, scar, or necrotic tissue, etc.

In one aspect, the invention features a method of imaging and sampling a tissue. The method includes inserting a catheter probe into a biopsy needle attachable to a hand-held scanning and sampling device and maneuvering the biopsy needle to an investigation site. In some embodiments, the biopsy needle is attachable to a tabletop scanning and sampling device. The method also includes capturing a three-dimensional image of the tissue at the investigation site.

In another aspect, the invention features a hand-held optical coherence tomography imaging and tissue sampling system. The system includes a hand-held unit including a handle and an elongated member. The system also includes a biopsy needle coupled to a distal end of the elongated member. An electromechanical device is coupled to a proximal end of the elongated member. The electromechanical device is configured to rotate and pull an optical coherence tomography probe within the biopsy needle so that a three-dimensional helical or axial scan of tissue is captured processing unit.

In another aspect, the invention features n optical coherence tomography probe The probe includes a single-mode optical fiber. A spacer (e.g., a coreless fiber) is coupled (e.g., fused) to the single-mode fiber and an optical element (e.g., a 45 deg polished ball lens) is coupled to the spacer. The optical element includes a gold coating on a surface of the optical element. In some embodiments the gold coating is reflective. The gold coating can be on a polished surface of the optical element.

The method can also include removing the catheter probe from the biopsy needle. An aspirate can be performed at the investigation site. The aspirate can be performed in situ.

In some embodiments, capturing the three-dimensional image is performed in situ. The three-dimensional image can be captured at a tip region of the biopsy needle. The three-dimensional image can include micron scale images of tissue morphology at the investigation site. In some embodiments capturing the three-dimensional image includes rotating and pulling the catheter probe within the biopsy needle to generate a three-dimensional helical or axial scan of the tissue at the investigation site.

A reflectivity profile or texture of the three-dimensional image, or both can be analyzed to determine a nature of the tissue and the investigation site. In some embodiments identifying, in real time, the nature of the tissue at the investigation site can be identified in real time. The nature of the tissue at the investigation site can be displayed to a user while maneuvering the biopsy needle. In some embodiments a sample of the tissue at the investigation site can be obtained.

In some embodiments, the catheter probe can be an optical coherence tomography probe. The optical coherence tomography probe can include an optical fiber and a protective transparent tube. The optical fiber can be disposed within the protective transparent tube. The protective transparent tube can include a nitinol tube with a polytetrafluoroethylene distal end. In some embodiments the protective transparent tube includes polytetrafluoroethylene. In some embodiments, the protective transparent tube is made solely from polytetrafluoroethylene.

The biopsy needle can be retracted to expose the catheter probe. The biopsy needle can be passed through an instrument channel of an endoscope. The biopsy needle can be about 5 feet in length. In some embodiments the biopsy needle can be about 6 feet in length. The biopsy needle can be a 19 gauge or a 22 gauge needle. Those of skill in the art recognize that other lengths can gauges of biopsy needles can be used based the specific application of the method and system.

In some embodiments, the processing unit is configured to capture and analyze the three-dimensional helical or axial scan of tissue. The processing unit can be configured to differentiate between normal tissue, scar tissue, necrotic tissue and tumor tissue.

In some embodiments, the probe also includes a protective transparent tube surrounding a segment of the optical coherence tomography probe.

A proximal end of the spacer (e.g. a coreless fiber) can be fused to an end of the single-mode fiber. The optical element can include one of a ball lens or a gradient-index fiber. The optical element can be fused to a distal end of the spacer. In some embodiments the ball lens is a 45 degree polished ball lens. The gold coating can be on a polished surface of the 45 degree polished ball lens.

Other aspects and advantages of the invention will become apparent from the following drawings and description, all of which illustrate principles of the invention, by way of example only.

### BRIEF DESCRIPTION OF THE DRAWINGS

The advantages of the invention described above, together with further advantages, may be better understood by referring to the following description taken in conjunction with the accompanying drawings, in which like reference characters refer to the same elements throughout the different views. The drawings are not necessarily to scale, emphasis instead generally being placed upon illustrating the principles of the invention.

FIG. 1 is a perspective view of a hand-held OCT scanning engine, according to an illustrative embodiment of the invention.

FIG. 2 is a detailed view of a fiber-optic rotary junction, according to an illustrative embodiment of the invention.

FIG. 3 is a schematic view of an imaging probe catheter assembly, according to an illustrative embodiment of the invention.

FIG. 4 is a detailed view of a tip portion of the imaging probe catheter of FIG. 3, according to an illustrative embodiment of the invention.

FIG. 5 is a detailed view of an optical element of the tip portion (FIG. 4) of the imaging probe catheter of FIG. 3, according to an illustrative embodiment of the invention.

FIG. 6 is a schematic view of a biopsy needle retraction mechanism, according to an illustrative embodiment of the invention.

FIG. 7 is a detailed view of a biopsy needle of FIG. 6, according to an illustrative embodiment of the invention.

FIG. 8 is a ZEMAX optical design of the imaging probe catheter assembly of FIG. 3, according to an illustrative embodiment of the invention.

FIG. 9 is a theoretical light spot diagram of the imaging probe catheter assembly of FIG. 3, according to an illustrative embodiment of the invention.

FIG. 10 is a diagram illustrating an optical design, according to an illustrative embodiment of the invention.

FIG. 11 is a diagram illustrating a through focus spot size as a function of a length of a gradient-index (“GRIN”) segment of fiber, according to an illustrative embodiment of the invention.

FIG. 12A is an OCT image of a normal colon, according to an illustrative embodiment of the invention.

FIG. 12B is a histology image of a normal colon, according to an illustrative embodiment of the invention.

FIG. 13A is an OCT image of adenocarcinoma in mouse colorectal region, according to an illustrative embodiment of the invention.

FIG. 13B is a histopathology image of adenocarcinoma in mouse colorectal region, according to an illustrative embodiment of the invention.

FIG. 14 is an image of exemplary processed and tissue-type assigned OCT data, according to an illustrative embodiment of the invention.

FIG. 15 is a schematic illustration of an OCT or Optical Coherence Microscopy (“OCM) system, according to an illustrative embodiment of the invention.

### DESCRIPTION OF THE INVENTION

It should be understood that when an element is referred to as being “on” or “connected” or “coupled” to another element, it can be directly on or above, or connected or coupled to, the other element or intervening elements can be present. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.). When an element is referred to herein as being “over” another element, it can be over or under the other element, and either directly coupled to the other element, or intervening elements may be present, or the elements may be spaced apart by a void or gap.

Embodiments of the present invention relate to the development of a high-resolution optical imaging probe that can be deployed within organs of the body through a minimally-invasive biopsy needle or directly through the instrument channel of an endoscope and which can be used to differentiate between normal, scar, necrotic, and tumor tissue. It is well known, that tissue differentiation is a very challenging problem, especially for deeply located organs, for example, lungs, liver, pancreas, etc. While traditional radiological imaging modalities allow for high depth imaging, their resolution is limited to hundreds of microns, which is not sufficient for tissue differentiation. Recently, optical imaging techniques, such as OCT, have proven that imaging of tissue morphology at the micron scale is possible, which enables differentiation between normal and abnormal tissue. Unfortunately, OCT penetration depth is limited to several millimeters. To image tissue located at higher depths (e.g., deeper in the body), minimally invasive OCT probes have been developed. However, their relatively large size (over 1 mm in diameter), contamination by body fluids, and limited imaging performance have been the main obstacles in expanding OCT use for imaging interstitial tissue. The development of a sub-millimeter fiber optic OCT probe that can reach any organ within the body and provide micron scale OCT images is needed. It can be encapsulated within a hypodermic tube so that the imaging optics does not get contaminated by body fluids.

OCT has proven its capability to image tissue morphology at the micron scale, and thus has enabled the diagnosis of cancer diseases in their early stage, at the epithelial level, as well as various cardio diseases. However, the use of OCT for imaging of interstitial tissue is somewhat limited due to the lack of minimally invasive probes that can be deployed alone, or by means of biopsy needles to image tissue morphology in various organs, where a natural lumen does not exist or cannot be used to image deeper within the organ. OCT applicability and translation to clinical use, especially for imaging interstitial tissue, has been limited by the lack of adequate probes that can be deployed safely without significantly disrupting tissue morphology, inducing bleeding, while providing high-quality images. Currently, there are no reports of high-resolution imaging OCT probes that can be deployed through fine gauge biopsy needles (e.g., 22 gauge, 19 gauge, or smaller), with a length of several inches to several feet. Thus, imaging of less accessible organs, such as pancreas, liver, or kidney has not yet been performed with such probes. A benefit of the proposed probe and imaging approach is that it can be used in correlation with the actual biopsy procedures, without adding any complications. For example, the imaging probe can be sent through the biopsy needle and used to examine tissue composition before an aspirate will be made. The biopsy needle, with the imaging probe inside it, can be moved into the organ until adequate representation of the investigated site can be obtained. Then the imaging probe can be removed and an aspirate, or biopsy suction, can be performed from the imaging location. Thus, this new biopsy-guidance approach has the potential to significantly improve the outcome of the biopsy procedures.

An approach for increasing biopsy success rate includes the use of a hand-held system composed of a three-dimensional (“3D”) scanning engine and a fiber optic-based catheter (e.g., an OCT probe) that can be inserted in regular biopsy needles and produce 3D images of tissue morphology at the needle tip. A biopsy needle, transcutaneously inserted in the body with an imaging probe inside, can be moved into an organ, for example, lungs, breast, pancreas, liver or kidney, until adequate representation of the investigated site is obtained. The imaging probe can be removed and an aspirate or biopsy suction can be performed from the imaging location. This biopsy-guidance approach has the potential to significantly improve the outcome of biopsy procedures.

Embodiments of the invention include a minimally invasive OCT probe that can be deployed through a long (e.g., about 5 to 6 feet) biopsy needle, 22 gauge or smaller, which is usually deployed into the body through the instrument channel of an endoscope and then inserted through the organ wall (eg. stomach, colon, vagina) into a close by organ of interest. The probe can provide micron-scale images of tissue morphology, is sterilizable, and the imaging optics do not require any additional protection to avoid contamination with blood or body fluids.

Embodiments also include a method, probe and system that allows for imaging tissue morphology, at the micron scale, through a biopsy needle. Applications of the OCT probe include imaging the morphology of the organs that are only accessible through a biopsy needle, transcutaneously, or combined endoscopic and trans-organ sapling. Thus, the probe can be used to assist and guide the biopsy process by providing high resolution images of the tissue at the biopsy site. This can benefit the investigation of the highly heterogeneous tissues, for which high rates of non-diagnostic biopsies occur. This is the case for breast, pancreas, liver, kidney or lung biopsies, and especially in patients with cancer recurrence, where surgical and/or radiation therapy scars and necrosis are present. Embodiments described herein can employ OCT and/or optical coherence microscopy (“OCM”) techniques.

Other embodiments include an optical design and configuration for a combined GRIN and microlens-based probe or a microlens alone with diffraction-limited imaging performance at different adjustable imaging depths. In addition, fusion of the optical elements of the OCT probe can ensure stability, ease of manufacturing, and mechanical robustness. Encapsulation of the OCT probe within a flexible tube can allow for imaging through long biopsy needles, accurate transmission of the scanning motion (axial or rotary), and chemical sterilization. The OCT probe can be placed at the biopsy site through the long biopsy needle (e.g., a 19 Gauge, Model M00550040, Boston Scientific biopsy needle, or similar).

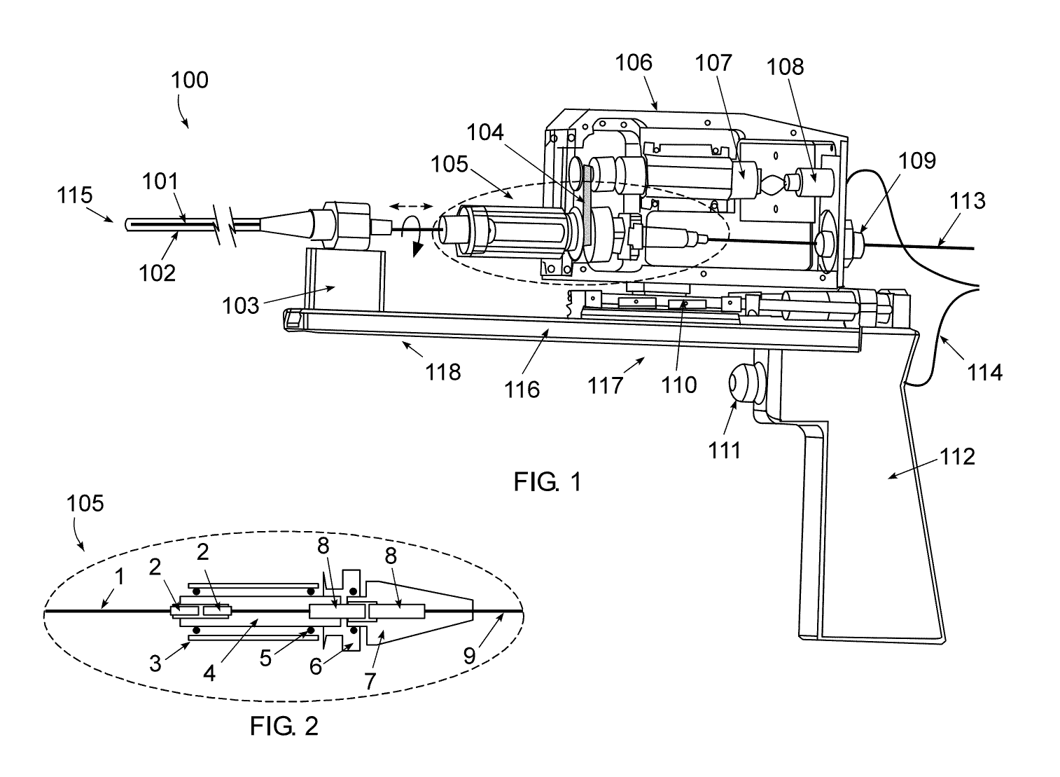

FIG. 1 is a perspective view of a hand-held OCT scanning engine 100 that includes a fiber optic catheter probe 101, for example, an OCT probe or an OCM probe. FIG. 2 is a detailed view of a fiber-optic rotary junction 105 of FIG. 1. The hand-held OCT scanning engine 100 includes a catheter probe 101, a protective sheath 102, a holder assembly 103 for the sheath tube 102, a belt 104, a fiber optic rotary junction (“FORJ”) 105, a FORJ body holder 106, a motor 107, an electrical connector 108, a fiber connector 109, a motorized stage 110, a FORJ switch 111, a handle 112, a single mode optical fiber 113, and an electrical wire 114. The FORJ 105 includes a catheter probe 1, and fiber connector 2, a body 3, a shaft 4, ball bearings 5, a pulley system 6, a rotary junction stator 7, a fiber pigtailed collimator 8, and a single mode optical fiber 9.

The hand-held OCT scanning engine 100 can image and sample tissue within the human body or an animal. The hand-held unit includes a handle 112 and an elongated member 116 having a proximal end 117 and a distal end 118. A biopsy needle (not shown) can be coupled to the holder assembly 103. The holder assembly 103, at the distal end 118 of the elongated member 116, can be used to couple a protective sheath 102, or directly to a biopsy needle. The catheter probe 101 can be passed through the holder assembly 103. The biopsy needle can contain the catheter probe 101 inside. An electromechanical device 119 that spins and translates the OCT catheter probe 101 can be coupled to the proximal end 117 of the elongated member 116. The electromechanical device 119 can include, for example, the belt 104, the FORJ 105, the FORJ body holder 106, the motor 107, the electrical connector 108, the fiber connector 109, and the motorized stage 110. The electromechanical device 119 can be configured to rotate, push and pull the catheter probe 101, e.g., an OCT or OCM probe, within the biopsy needle (not shown) so that a 3D helical or axial scan of the tissue can be captured at a processing unit (not shown).

As described above, the catheter probe 101 can be encapsulated or surrounded by a protective sheath 102. In this embodiment, the catheter probe 101 is disposed within an optically transparent protective sheath 102, which can be coupled to the holder assembly 103. The catheter probe 101, 1 can be coupled to the FORJ 105 by a fiber connector 2.

As described above, the hand-held OCT scanning engine can be configured with push and pull-back capabilities as well as rotational capabilities. That is, the hand-held OCT scanning engine can be configured to push, pull and/or rotate the probe 101. To provide the push and pull-back capabilities, the FORJ body 106 is coupled to the motorized stage 110. To provide the rotational capabilities, a motor 107 transmits the rotary motion through the FORJ 105 to the fiber connector 2 via a shaft 4 and pulley 6. The fiber connector 2 is disposed within a body 3 and rests on ball bearings 5. A pair of fiber pigtailed collimator 8 can be used for coupling a radiation beam from a stationary fiber optic 113 attached to the connector 109 to catheter probe 101, which is attached to the mobile part of the FORJ 105, specifically to connector 2.

The push, pull-back and rotational capabilities allow the hand-held OCT scanning engine 100 to generate helical tissue scans. In this manner, the scanning engine can scan a larger volume of tissue and increase the probability of detecting areas of vital tumor tissue. For example, considering an average imaging depth of 1.5 mm and a pull-back of 5 mm to 10 mm distance, a cylindrical volume of about 3 mm×5 mm to 20 mm can be investigated. This surpasses the capabilities of any existing biopsy approach. For example, in one embodiment the hand-held OCT scanning engine is configured to with an axial imaging resolution of 6 μm, a lateral imaging resolution of 10 μm, an imaging speed of 50 fps, an imaging depth of up to 2 mm, and a pull-back scanning range of 20 mm.

The fiberoptic part of the OCT catheter 101 can be encapsulated within a nitinol (i.e., nickel titanium) tube 102, with the distal end 115 open to expose the curved surface of a ball lens (not shown), or within a torque coil. In either case, the proximal end is attached to a fiber optic connector.

The protective sheath 102 can be a nitinol (i.e., nickel titanium) tube with an optically transparent distal end 115 made of polytetrafluorethylene (“PTFE”) (e.g., Teflon®). The protective sheath 102 can be made entirely of PTFE or other optically transparent material and can be dimensioned to fit within the inner diameter of a biopsy needle. The protective sheath 102 (e.g., a protective transparent tube) can be made from a combination of optically transparent materials, which are known to those of skill in the art. In some embodiments, the protective sheath 102 is flexible to allow for imaging through long biopsy needles, accurate transmission of the scanning motion, and chemical sterilization.

The protective sheath 102 can be coupled to a holder assembly 103, which is in turn coupled to the handle 112 for easy manipulation and control by a user. Referring to FIG. 2, the catheter probe 1 can be coupled to the FORJ 105 by a fiber connector 2, which is coupled to a body 3. The OCT probe 101 can include an optical fiber 113, 9. The optical catheter 101 can be at least partially disposed within the protective transparent tube 102.

The FORJ 105, coupled to an FORJ body holder 106, can include a pair of two collimators, e.g., fiber pigtailed collimator 8, to narrow a propagation of waves traveling through the device. The collimators can be, for example, a curved mirror or lens. A micro-motor 107 can be provided to rotate the catheter probe 101, so a rotary scan can be obtained. The FORJ 105 has a stator 7 with a pigtailed collimator 8. This part of the FORJ 105 is attached to the hand-held probe case 106. The FORJ rotor includes a pulley 6 attached to a sleeve 4 that holds a short segment of fiber 9 terminated with a micro connector 2 at one end and a collimator 8 at the other end. This assembly rotates inside of a holder 3 attached to the probe body 106. Ball bearings are used to hold the sleeve 4 inside of the holder 3 while allowing for rotational motion. The entire assembly 106 can be attached to a translation stage 110 to provide a translational motion of the catheter 101, which together with the rotary motion generate a 3D helical scan. The speed of the micro-motor 107 can be adjusted by a controller while an optocoupler can be used to read the position of the FORJ 105 shaft and initialize a scanning engine to always start at the same angle and sync the rotary scan with a computer data display rate. The same approach can be taken with the translational stage 110, such that a user can position the catheter probe 101 at any desired location within the tissue and search for specific features of a sample at an investigation site. The motorized stage 110 can be used to pull back the catheter probe 101 while the micro-motor 107, facilitated by belt 104 and FORJ 105, rotates the catheter probe 101 to generate a 3D helical scan. The speed of the motorized stage 110 can also be adjusted by the controller, which allows a user to modify the density of the helical scan. An electrical switch 111 can be used to start/stop the rotation of the motor 107. Electrical wires, 114 connect the switch 111 and the motor 107 to a controller. A fiber optic connector 109 can be used to optically connect the hand-held probe to the OCT engine through a single-mode fiber optic patchcord.

The hand-held OCT scanning engine 100 can be used to image and sample a tissue. The method includes inserting the catheter probe 101 into a biopsy needle. The biopsy needle can be attached to the hand-held scanning and sampling device, for example, the hand-held OCT scanning engine 100. A user can maneuver the biopsy needle to an investigation site. The investigation site can be any tissue or organ in an animal or human, for example, the lungs, liver, or kidneys. The catheter probe 101, for example an OCT or OCM probe, can capture a 3D helical or axial image of the tissue at the investigation site. The 3D image capture can be performed in situ. Instead of, or in addition to, capturing a 3D image of the tissue, a sample of the tissue can be obtained at the investigation site. Obtaining a sample of the tissue can allow the user to perform further tests on the tissue.

To capture the 3D image, the catheter probe 101, which is within the biopsy needle, can be rotated, pushed and/or pulled to generate a 3D helical or axial scan of the tissue at the investigation site. The electromechanical device 119 can rotate and pull the catheter probe from the distal end 118 to the proximal end 117 of the elongated member 116. The 3D image can include micron scale images of tissue morphology at the investigation site. The micron scale resolution images (e.g., 6 to 10 microns obtained over a 3D volume with a 3 mm diameter and a length of 5 to 20 mm allow for tissue differentiation, unlike the traditional radiological imaging modalities which do not have sufficient resolution (e.g., hundreds of microns) for tissue differentiation.

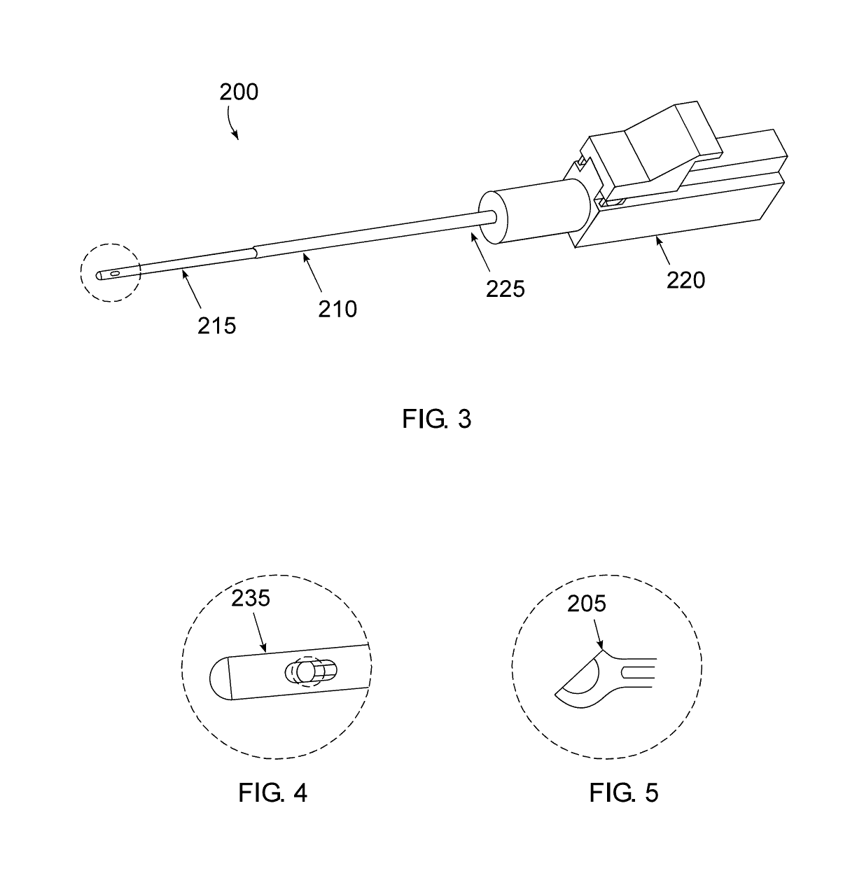

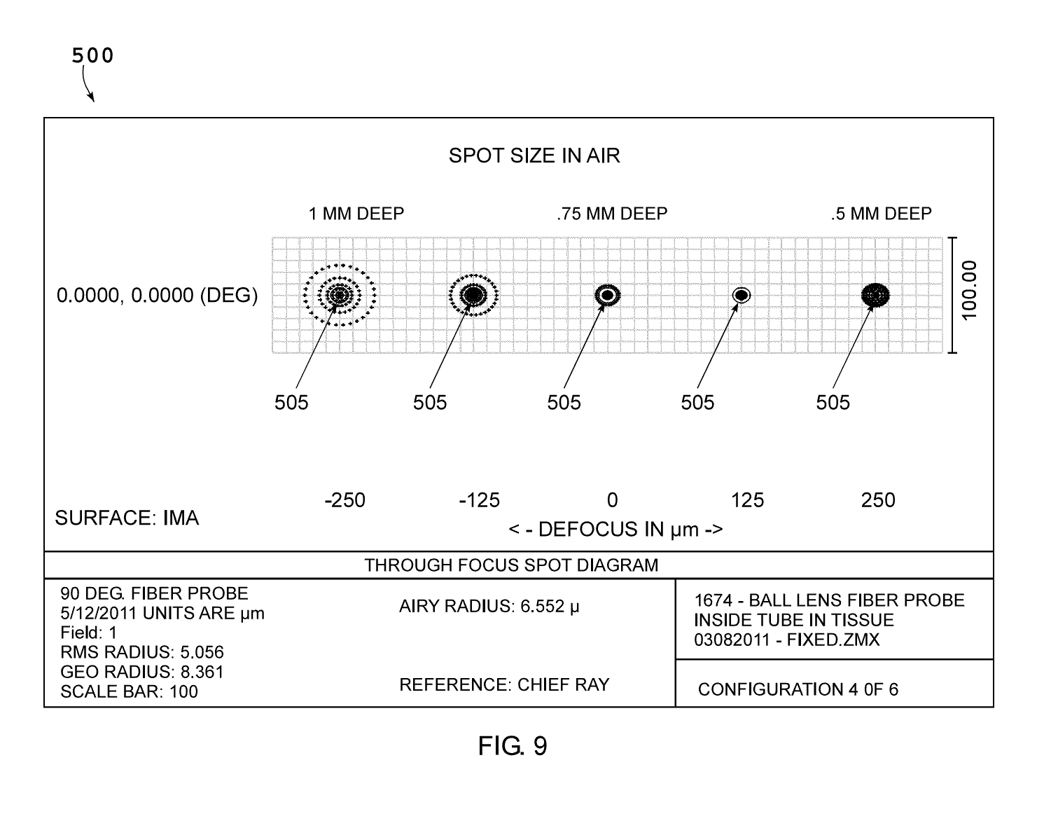

FIG. 3 is a schematic view of an imaging probe catheter assembly 200. FIG. 4 is a detailed view of a tip portion 235 of the imaging probe catheter of FIG. 3 and FIG. 5 is a detailed view of an optical element 205 of the tip portion (FIG. 4) of the imaging probe catheter of FIG. 3. The imaging catheter assembly 200 can be based on a fiber optic design and can be encapsulated or surrounded, wholly or partially, within a small size (e.g., about 280 μm to about 500 μm inner diameter) hypodermic tube that can have a length up to several feet and can be deployed into the organ of interest through a regular biopsy needle. It can allow for near diffraction-limited imaging (15-30 μm Airy disk) of the tissue at a wavelength of 1310 nm; however, other wavelengths can be used. The imaging optics can include a small segment of coreless fiber or spacer, which is fused on one side to a single-mode fiber (e.g., the single mode optical fiber 113 of FIG. 1 or 9 of FIG. 2), and on the other side to an optical element, e.g., a 45° polished lens 205 or a GRIN fiber. The lens 205 can provide the necessary optical power for imaging. These optical elements can be fused together to eliminate, or nearly eliminate, back-reflections from the optical interfaces that can be present in the OCT image. The position of the focal plane and the diameter of the optical beam in this plane can be adjusted by modifying the length of a coreless segment of optical fiber and the diameter of the ball lens 205.

A nitinol tube 210 can be used to encapsulate or surround the catheter probe 215 and a fiber optic connector 220 can be attached to the proximal end 225 of the tube 210. A distal end 230 of the catheter probe 215 (e.g., the catheter probe 101 of FIG. 1) has a tip 235 (shown in the enlarged image of FIG. 2) where the polished lens 205 can be housed. The tube 210 surrounds the catheter probe 215 and the polished lens 205. As discussed above, the catheter probe 215 can be an OCT probe. The 3D image captured by the probe 215 can be captured at the tip 235 of the probe 235 and/or biopsy needle (not shown).

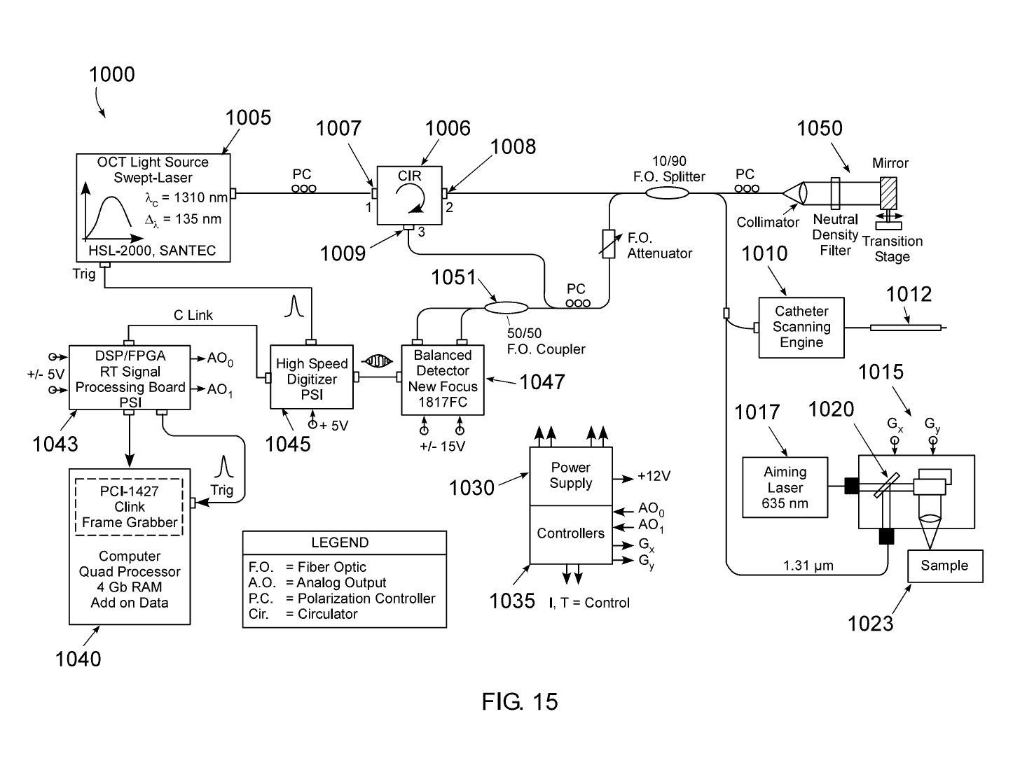

The tube 210 can be a supereleastic hypodermic tube of medical grade. The fiber optic connector 220 can be a Diamond E 2000 fiber optic connector which can be used to connect the catheter probe 215 to an OCT system, such as the OCT system of FIG. 15 (below). The catheter can be approximately seven feet in length and can be deployed into an organ through a regular 22 or 19 gauge (or smaller) biopsy needle. To avoid any fiber breakage the entire assembly can be sealed with a biocompatible epoxy (e.g., EP-30-MED, Master Bond, USA). This design can allow for easy sterilization with ethylene oxide (EtO) EtO sterilization is mainly used to sterilize medical and pharmaceutical products that incorporate electronic components, plastic packaging or plastic containers.

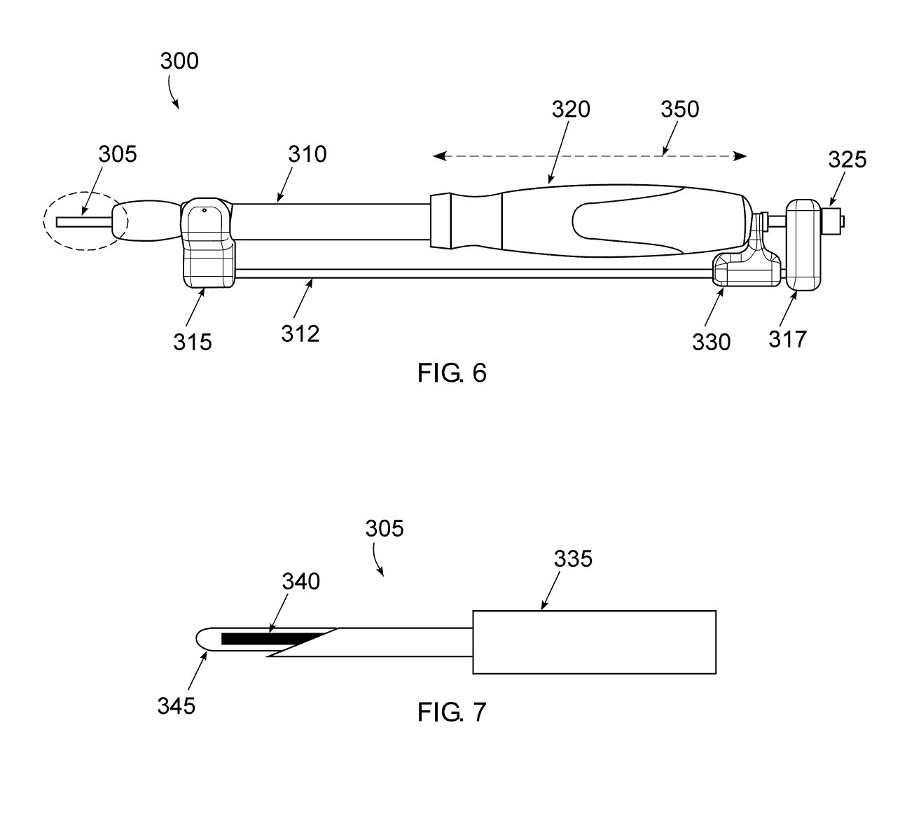

FIG. 6 is a schematic view of a biopsy needle retraction mechanism 300. FIG. 7 is a detailed view of a biopsy needle of FIG. 6 The needle retraction mechanism 300 includes a biopsy needle 305, a first slider 310, a second slider 312, a first holder 315, and second holder 317, a handle 320, a connector 325 (e.g., a Luer lock), and a guiding piece 330. In combination, the first slider 310, the first holder 315, the second slider 312, the second holder 317 and the guiding piece 330. By moving the handle 320, the needle 305 can move inside the jacket 335, while the catheter protective tube can be held in the same position, being attached to holder 325. In this way, after inserting the needle 305 into the tissue (e.g, with a stylet inside), the stylet can be removed and the OCT catheter (within the protective tube) can be inserted into the needle 305 until reaching the needle tip. The connector 325 can lock the catheter sheath 345 in place after being inserted into the needle 305. Then, the needle 305 can be slightly retracted by pulling the handle 320 to expose the OCT catheter 340 to tissue. The catheter 340 is still protected by the protective tube 345. The whole assembly can be attached to the proximal end of an imaging endoscope. Thus, by holding the endoscope, one can move the handle 320 and expose the OCT catheter 340. The OCT catheter 340 can be then rotated and pulled back inside the protective tube 345 to generate a 3D scan. The handle 320 can slide back and forth (see the double sided arrow 350) along the first slider 310 over about three inches in length. In some embodiments the handle 320 can slide less than three inches in length, for example one or two inches in length, or the handle 320 can slide more than three inches in length, for example, four or five inches in length. As a user slides the handle 320, the needle 305 moves in and out of the needle cover allowing the catheter 340, e.g., an OCT probe, to capture an image. The OCT probe can capture a 3D axial image, or a 3D helical image of an investigation site.

In some embodiments, the biopsy needle 305 can be passed through an instrument channel of an endoscope (not shown). An endoscope can be used to examine the interior of a hollow organ or cavity and can be inserted directly into an organ. Therefore, when the biopsy needle 305 is used in combination with an endoscope, a 3D image of the inside of an organ or cavity can be obtained.

The biopsy needle 305 can be long, for example, the biopsy needle 305 can be about 5 feet in length. The biopsy needle 305 can be any length suitable for the site to be investigated in the patient, for example, the biopsy needle 305 can be about 3 feet, 4 feet, 6 feet, 7 feet, or any increment in between. In some embodiments, the biopsy needle can be short, for example about 6 inches in length. The biopsy needle 305 can be a 19 or 22 gauge needle or any other size that is sufficient to house the catheter probe 340.

As discussed above with respect to FIG. 1, the hand-held OCT scanning engine 100 can be used to image and sample a tissue. Referring to FIGS. 6 and 7, the catheter probe 340 can be removed from the biopsy needle 305. This can allow an aspirate to be performed at the investigation site, e.g., the site where the tissue is being imaged and/or sampled. The aspirate can be performed in situ. The biopsy needle 305 can be retracted to expose the catheter probe.

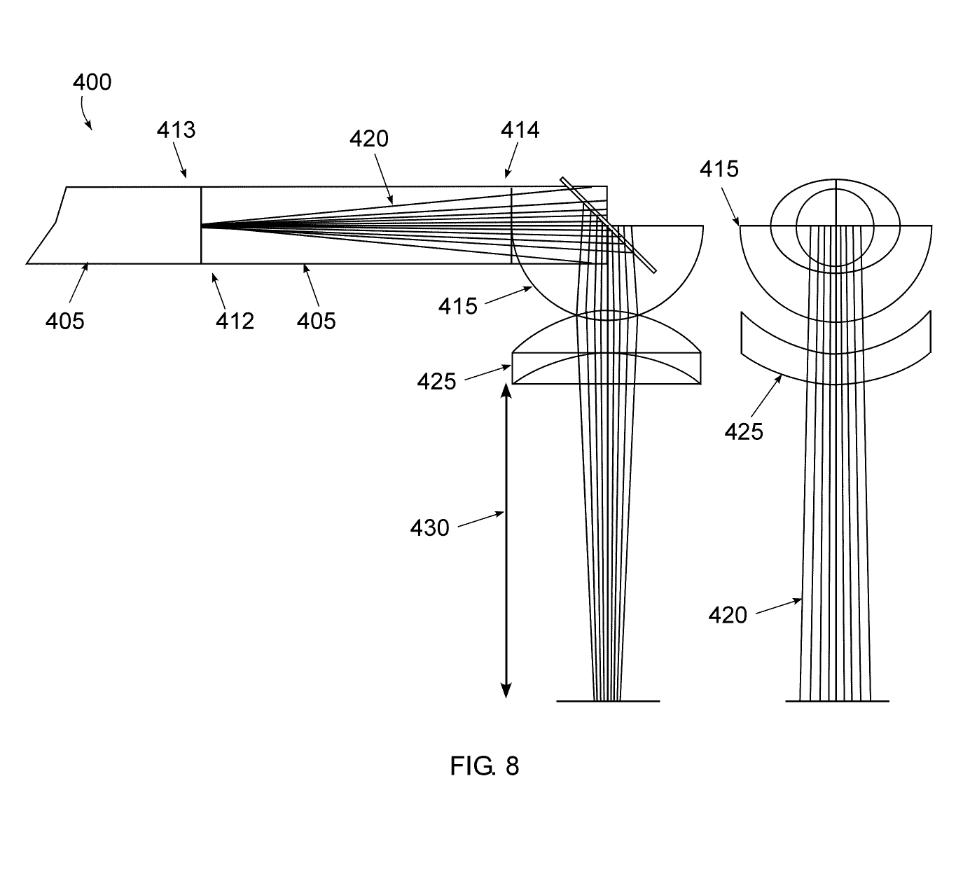

FIG. 8 is a ZEMAX optical design 400 of the imaging probe catheter assembly (e.g., an OCT probe) 200 of FIG. 3. A ZEMAX design (i.e., an optical design program sold by Radiant Zemax, LLC of Redmond Wash.) can be performed to optimize the parameters of the probe 200 and correct for various aberrations that can be induced by the protective sheath 210, e.g., astigmatism. The ZEMAX design models the propagation of rays through optical elements such as a lens as well as models the effect of optical coatings on surfaces of components.

As shown in FIG. 8, the imaging probe catheter, or OCT probe, includes a single mode optical fiber 405, for example SMF-28® fiber made by Corning® Incorporated of Corning, N.Y. The single mode fiber 405 can be coupled to a spacer 410, e.g., a coreless fiber. In some embodiments, a proximal end 412 of the spacer 410 is fused to an end 413 of the single mode fiber 405. The spacer 410 can be silica glass that has the same diameter as the single mode fiber 405. The diameter of the spacer 410 and single mode fiber 405 can be about 125 microns, e.g., the core size diameter of the single mode fiber 405. The length of the spacer 410 can be about 200 microns to about 500 microns depending on the desired focal length 430. The spacer 410 allows the beam 420 diameter to diverge so that the maximum optical power of the single mode fiber 405 can be realized. The spacer 410 can be coupled (e.g., fused) to an optical element, for example a ball lens 415 or a GRIN fiber (not shown). The optical element (e.g., the ball lens 415) can be fused to a distal end 414 of the spacer 410. The ball lens 415 can be a 45° polished lens. The optical element (e.g., the ball lens 415 or GRIN fiber) can include a gold coating on a surface of the optical element. The spacer 410 can be fused to a glass segment of a 45° polished ball lens. The gold coating can be on the polished surface of the 45° polished ball lens. A GRIN fiber can be used instead of a ball lens 415 when, for example, forward imaging (e.g., capturing a helical scan) is desired. For a forward image, no rotation of the catheter probe is required. Illustrative dimensions of the coreless fiber or spacer 410 and the ball lens 415 are shown in Table 1.

TABLE 1

| Portion | Dimension (mm) |
|---|---|
| Spacer 410 | 0.500 |
| Ball Lens 415 | 0.150 |

The ZEMAX optical design 400 models the propagation of rays (e.g., beam) 420 through the spacer 410, ball lens 415 and protective transparent tube 425. The protective transparent tube 425 (orthogonal views) can surround the OCT probe to protect it from being contaminated by body fluids. However, this tube inserts some aberrations of the outgoing beam (cylindrical lens effect), which causes the rays 420 to diverge into a focal point at a certain depth in the tissue 430. In some embodiments, the propagation of rays 420 through an optical element (e.g., corrective lens) can be implemented to correct this effect.

FIG. 9 is a theoretical light spot diagram 500 of the imaging probe catheter assembly 200 of FIG. 3. The light spot diagram 500 is produced by the ZEMAX optical design 400 of FIG. 8. The light spot diagram 500 is a collection of points, representing the distribution of the delivered optical power in the focal plane (situated at 0.60 mm away from the ball lens surface) (e.g., rays 420 of FIG. 8) when propagating through the fiber optic system. The Airy disk radius 505 (e.g., the best focused spot of light that a perfect lens can make) of 6.552 μm is shown in the spot diagram 500 as the solid circle. As can be seen in FIG. 8, while moving away from the focal plane, this spot becomes bigger because the optical beam starts diverging again after passing the focal plane. However, even at 1 mm away from the ball lens surface a large fraction of the beam energy is distributed within the Airy disk. Based on these observations, the distance between the probe and tissue has to be properly adjusted to get optimum performance.

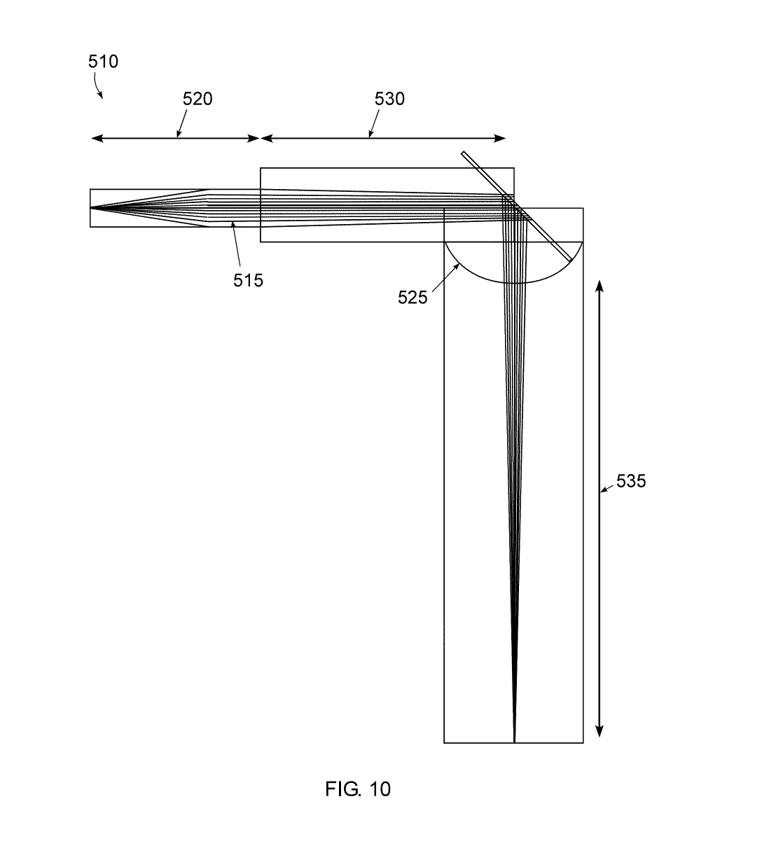

FIG. 10 a diagram illustrating a slightly different optical design of the probe 510, which is appropriate to use in those cases where a protective transparent tube cannot be used to protect the ball lens and keep it within the air. In those cases, the ball lens will be in direct contact with tissue and body fluids, which have a refractive index close to that of the ball lens. Therefore, the optical power of the lens decreases significantly. Therefore, in one embodiment, an imaging beam 515 is launched into a single-mode optical fiber (not shown) that is terminated with a micro-objective including a gradient-index (GRIN) fiber 520 and microlens or ball lens 525.

The main focusing power in this case comes from the GRIN fiber 520, which has its length calculated to focus the beam 515 into a very small spot, for example at about 750 microns depth in tissue. However, since the beam 515 needs to be scanned to create an image, it has to be deflected laterally. Therefore, a 45 deg polished ball lens 525 is attached to the GRIN fiber to deflect the beam laterally. Alternatively, a coreless fiber segment 530 can be polished at 90 degrees; however, it may become more susceptible to breakage due to sharp edges. Although the lens has a high optical power in air, when in contact with tissue or index matching fluid its power is dramatically reduced. Therefore, in some embodiments, the design is to create most of the focusing power by the GRIN fiber 520. The ball lens 525 can be made of coreless fiber to avoid inducing of astigmatism. In the case of a GRIN fiber used in place of the ball lens 525, the astigmatism can be sever and degrade image quality. The focusing parameters can be adjusted by modifying the length of the GRIN fiber 520. Table 2 shows the lengths of the GRIN fiber 520, the coreless segment 530, and the focal depth 535. The polished surface of the lens or coreless segment can be gold covered to deflect the beam laterally. Without reflective coating, the beam will refract through this surface and go forward within the tissue.

TABLE 2

| Component | Length |
|---|---|
| GRIN Fiber 520 | 0.267 mm |
| Coreless segment 530 | 0.400 mm |
| Focal depth 535 | 0.750 mm |

Alternatively, if the probe can be inserted in a protective sheath without immersion fluid, the GRIN segment can be removed and the ball lens can be designed to create the optical power of the catheter. In this case, the polished surface can be coated with an anti-reflection coating to reduce back reflections, while the polished surface can be gold coating protected. If not gold covered, it instead can be polished at exactly 45 deg to meet the total internal reflection condition and thus to have the beam deflected laterally.

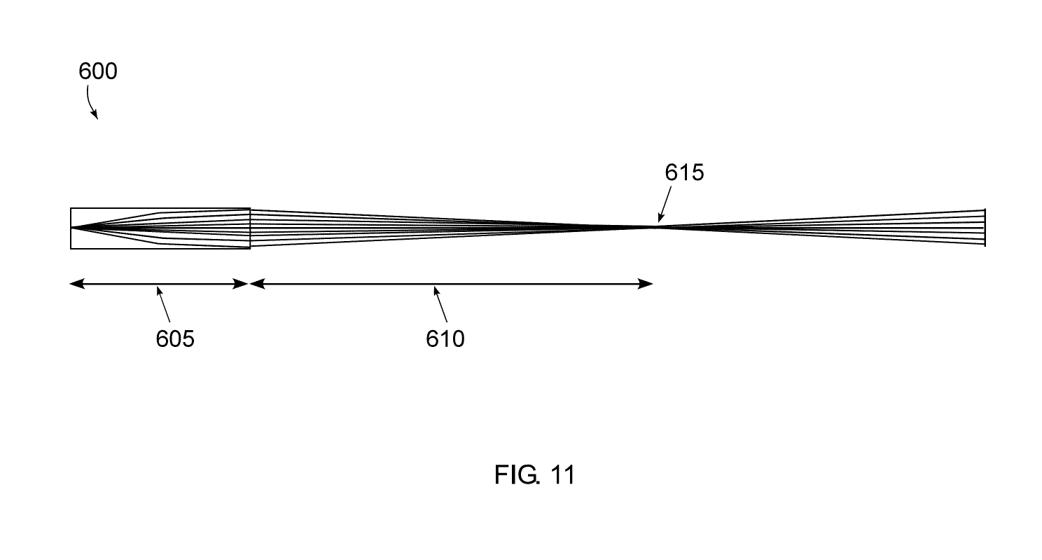

FIG. 11 is a diagram 600 illustrating a through focus spot size as a function of a length of a gradient index (“GRIN”) segment of fiber 605. The length of the GRIN fiber 605 is critical to the imaging performance of the catheter probe (e.g., catheter probe 101 of FIG. 1). In some embodiments, the length of the GRIN fiber 605 is computer controlled during the fabrication process. A fiber splicing instrument can be controlled by a computer that calculates the length of the GRIN fiber 605 as a function of the desired imaging parameters, such as spot size and imaging plane.

As shown in FIG. 11 and Table 3, diffraction limited performance within a Airy disc radius of about 30 μm (e.g., at about 1310 nm incoming beam) can be achieved at a focal position 610 (in air) of about 0.6 mm. The longer the focal plane, the larger the size of the focal radius 615. On the other hand, although the focal radius 615 improves (e.g., becomes smaller) with the decreasing of the focal position 610, the Rayleigh distance also becomes smaller. As a result, these parameters can be optimized as a function of the specific application. When a ball lens in contact with tissue is added, the focal position 610 (e.g., focal spot) is slightly affected by the radius of the ball lens. Therefore, the length of the GRIN segment 605 (e.g., spot size) needs to be further optimized.

TABLE 3
Note - Radius is Airy disc radius at 1^{st} zero.

| GRIN Length 605 | Focal Position 610 | Focal Radius 615 |
|---|---|---|
| <0.250 mm | Infinity | >220 μm |
| 0.255 mm | 3.687 mm | 76.9 μm |
| 0.260 mm | 1.856 mm | 42.4 μm |
| 0.265 mm | 1.218 mm | 29.3 μm |
| 0.270 mm | 0.903 mm | 22.4 μm |
| 0.275 mm | 0.716 mm | 18.1 μm |
| 0.280 mm | 0.591 mm | 15.2 μm |
| 0.285 mm | 0.503 mm | 13.2 μm |

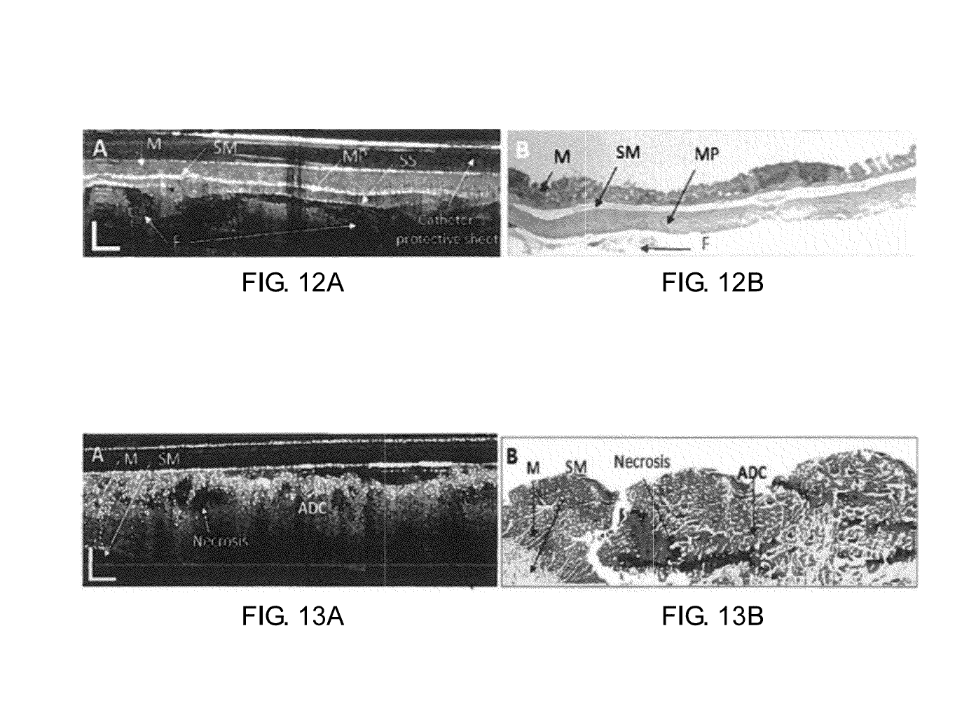

FIG. 12A is an OCT image of a normal colon, and FIG. 12B is a histology image of a normal colon. Normal morphology of the colon wall was observed in wild mice, as well as in 2 of the 8 week old APC mice (genetically-modified APC min+mice that develop colorectal cancers). The histology image (FIG. 12B) shows the mucosa (M), submucosa (SM), muscularis propria (MP) layer, with some underlying fat (F). The OCT image (FIG. 12A) displays the normal mucosa (M), the thin submucosal (SM) layer, the muscularis propria (MP), and the subserosa (SS) boundary with some underlying fat (F) or connective tissue layers. (OCT scale bar: 500 μm; Histology magnification: 5×.)

The subserosa (SS) is a layer of tissue between the muscularis and serosa, which sometimes is admixed with some fat (F). The submucosal (SM) layer was measured to be very thin in histology and not very well resolved in some of the OCT images. However, most of tissue boundaries remained clear and distinct at all depths, rotations, and time points. A highly scattered layer is observed on the top of the OCT image (FIG. 12A) This is the OCT catheter protective sheath, which can be made of Teflon and have a thickness of about 100 μm.

FIG. 13A is an OCT image of adenocarcinoma in mouse colorectal region, and FIG. 13B is a histopathology image of adenocarcinoma in mouse colorectal region. Mice with colorectal adeno-carcinoma were imaged. A representative case is shown in FIGS. 13A and 13B. The OCT image (FIG. 13A) shows the loss of tissue texture (absence of normal layers) and presence of pockets of uneven echoic or dark areas, which are caused by high signal absorption in the necrotic tissue or glandular destruction. As observed, adenocarcinoma (ADC) was invading and replacing the mucosa (M). These results demonstrate the imaging probes capability to reliably separate normal structures of the colon as well as its capability to detect alterations that correlate well with histology.

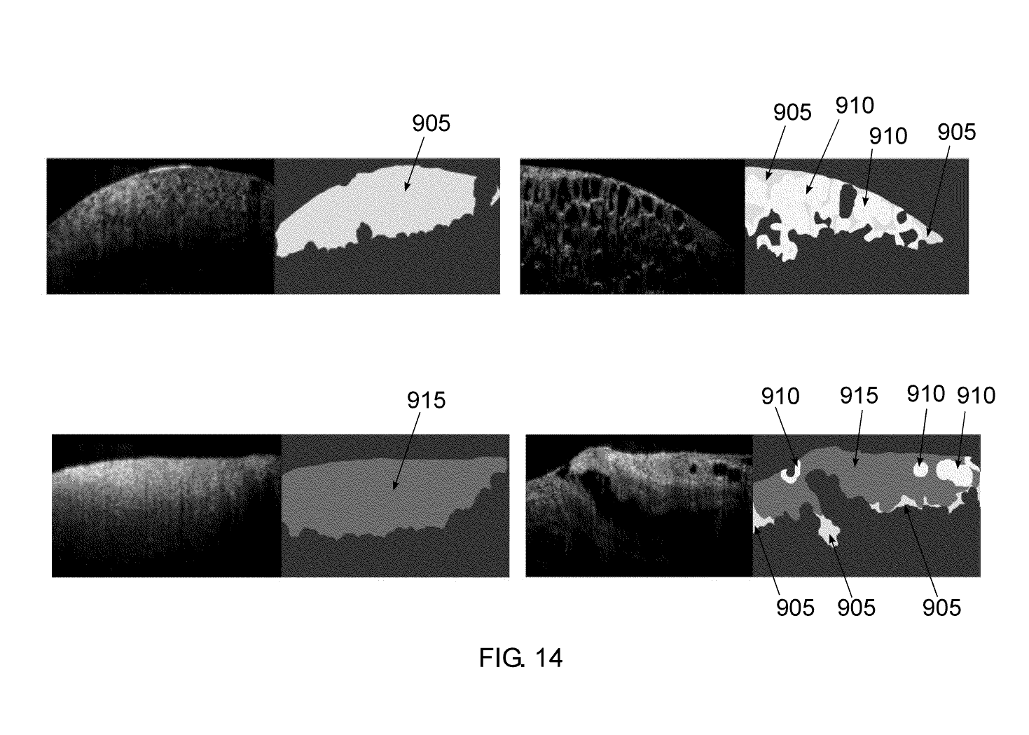

FIG. 14 is an image of exemplary processed and tissue-type assigned OCT data. The images on the left of each of the four image sets are OCT images, while images on the right of each of the four image sets are the processed data by an algorithm (discussed below) that can attribute a color to each tissue-type. For example, the fibrous tissue 905 can represented in light blue, the adipose tissue 910 in yellow, and the tumor 915 is in red. Any color can be assigned to the different tissue types. As discussed in more detail below, algorithms for tissue differentiation can be implemented on a parallel processing architecture, such as a General Purpose-Graphic Processing Unit to enable real-time tissue differentiation and provide clinician feedback to aid diagnosis.

FIG. 115 is a schematic illustration of an OCT or OCM system 1000. An OCT or OCM system can improve axial resolution and provide easy use and maneuverability in a clinical room. A large bandwidth light source 1005 (e.g., 150 nm bandwidth) can be used to provide an axial resolution in the order of 6 μm, which can help the system to detect smaller tissue features at the cellular level and thus to increase the probability of correct diagnosis. The light source 1005 can be in communication with a circulator 1006, which transmits the light output from the light source 1005 from a first port 1007 to a second port 1008 to various other components in the OCT or OCM system 1000, as well as the retro-reflected light from those elements from the second port 1008 to a third port 1009 and thus minimize losses in the system.

A catheter scanning engine 1010, for example, the hand-held OCT scanning engine 100 of FIG. 1, can be connected to the OCT or OCM system 1000 to perform various scanning and tissue sampling procedures. A fiber optic catheter 1012, described in more detail in FIG. 1 can be attached to the catheter scanning engine 1010. For testing tissue specimens, a benchtop scanning engine 1015 can be used instead of the hand-held catheter scanning engine described above. This scanning engine 1015 can be in communication with an aiming laser 1017 that can operate at about 635 nm. A dichroic mirror 1020 can be used to combine the beams from the aiming laser 1017 and OCT system so that laser beams hit the sample 1023 in exactly the same position.

The light coming back from the sample and from the optical delay line 1050 is combined together in a fiberoptic beam splitting/combining element to create interference fringes, which are sent to a detection scheme 1047 (balanced detector) through the second and third ports 1008, 1009 of the circulator 1006. To take benefit of the balanced detector scheme, which helps to reduce the influence of the light source intensity noise, a small fraction of the light coming from the optical delay line 1050 is combined with that coming from third port 1009 of the circulator 1006 by a 50/50 coupler 1051 and then sent to the inputs of the balanced detector 1047. After converting the optical signal into an electric signal a digtizer 1045 is used to convert the analog signal into a digital one. This signal can be sent to a real-time FPGA processing board 1043, and then to a frame grabber residing in the computer 1040, or directly to the frame grabber and perform the real-time processing of the signal into a General Purpose-Graphic Processing Unit (GP-GPU) residing in the computer 1040.

The OCT or OCM system 1000 also includes a power supply 1030 that provides power to the system 1000 and controllers 1035, which can control, for example, the micro-motor 107 of FIG. 1 or the galvanometers from the benchtop scanning engine 1015.

In some embodiments, algorithms for tissue differentiation can be implemented on a parallel processing architecture, such as a General Purpose-Graphic Processing Unit (GP-GPU) that will also reside in the computer 1040. The GP-GPU can enable real-time tissue differentiation and provide clinician feedback to aid diagnosis. Thus, the GP-GPU can be used to indentify, in real time, the nature of the tissue at the investigation site and display to the user the nature of the tissue at the investigation site while maneuvering the biopsy needle. The GP-GPU can either analyze a reflectivity profile or texture of the 3D image, or both, to determine the nature of the tissue at the investigation site. Advanced algorithms can be implemented into the GP-GPU to differentiate between normal tissue, scar tissue, necrotic tissue, tumor tissue, or any other type of tissue relevant to a user.

For example, the algorithms can create images that assign a color to specific tissue types (see, FIG. 14). The collected OCT images can be analyzed in real-time using an OCT system configured with various software algorithms, which can provide an estimate of the nature of the tissue present at the tip of the needle. These algorithms compare specific features of each tissue type (e.g., adipose, fibrous, cancer, scar, or necrotic), which are stored in a database, with current features of the investigated tissue. The analysis of each reflectivity profile in the acquired image can be performed along with a texture analysis. The mean values of the reflectivity profile parameters (e.g., slope, variance, spatial frequency of the peaks, etc.) and texture parameters (e.g., roughness, variance, and waviness) are used to build up a covariance matrix (see EQN 1),

$${S}_{i} = \frac{1}{{n}_{i}} {\sum }_{j = 1}^{{n}_{i}} ( {x}_{i , j} - \overline{{x}_{i}} ) {( {x}_{i , j} - \overline{{x}_{i}} )}^{T} \qquad EQN 1$$

where ni is the number of elements in each tissue class within the training set, and the superscript T indicates matrix transpose. The mean values for each parameter used in the algorithm, correspond to the five tissue types in the training set: adipose, fibrous, scar, tumor, and necrotic.

To determine the nature of a tissue that will be investigated, the mean values of the same parameters can be used to calculate covariant matrices and use them for calculating a quadratic discrimination score (see EQN 2),

dᵢ^{Q}=−½ ln |Sᵢ|−½(x− x̅ᵢ̅)^{T}Sᵢ⁻¹(x− x̅ᵢ̅) EQN 2

where |.| stands for determinant, T is transpose, Sᵢ and Sᵢ⁻¹ are the direct and inverse covariance matrices, xᵢⱼ and x are the column vector of the test and validation set parameters corresponding to the investigated tissue classes. The maximum quadratic score can be used to assign each investigated sample to a correct tissue type.

Although various aspects of the disclosed methods, devices and systems have been shown and described, modifications may occur to those skilled in the art upon reading the specification. The present application includes such modifications and is limited only by the scope of the claims.

What is claimed is:

1. A method of imaging and sampling a tissue, the method comprising:
inserting a catheter probe into a biopsy needle attachable to a hand-held scanning and sampling device;
maneuvering the biopsy needle to an investigation site; and
capturing a three-dimensional image of the tissue at the investigation site with the catheter probe.

2. The method of claim 1, further comprising removing the catheter probe from the biopsy needle.

3. The method of claim 2, further comprising performing an aspirate at the investigation site.

4. The method of claim 3, wherein performing the aspirate at the investigation site is performed in situ.

5. The method of claim 1, wherein capturing the three-dimensional image is performed in situ.

6. The method of claim 1, wherein the three-dimensional image is captured at a tip region of the biopsy needle.

7. The method of claim 1, wherein the three-dimensional image includes micron scale images of tissue morphology at the investigation site.

8. The method of claim 1, wherein capturing the three-dimensional image further comprises:
rotating and pulling the catheter probe within the biopsy needle to generate a three-dimensional helical or axial scan of the tissue at the investigation site.

9. The method of claim 1, further comprising analyzing a reflectivity profile and texture of the three-dimensional image to determine a nature of the tissue at the investigation site.

10. The method of claim 1, further comprising:
identifying, in real time, a nature of the tissue at the investigation site; and
displaying, to a user, the nature of the tissue at the investigation site while maneuvering the biopsy needle.

11. The method of claim 1, further comprising obtaining a sample of the tissue at the investigation site.

12. The method of claim 1, wherein the catheter probe is an optical coherence tomography probe.

13. The method of claim 1, further comprising retracting the biopsy needle to expose the catheter probe.

14. The method of claim 1, further comprising passing the biopsy needle through an instrument channel of an endoscope.

15. A hand-held optical coherence tomography imaging and tissue sampling system comprising:
a hand-held unit including a handle and an elongated member;
a biopsy needle coupled to a distal end of the elongated member; and
an electromechanical device coupled to a proximal end of the elongated member, the electromechanical device configured to rotate and pull an optical coherence tomography probe within the biopsy needle so that a three-dimensional helical or axial scan of tissue performed by the optical coherence tomography probe is captured at a processing unit.

16. The system of claim 15, wherein the processing unit is configured to capture and analyze the three-dimensional helical or axial scan of the tissue.

17. The system of claim 16, wherein the processing unit is configured to differentiate between normal tissue, scar tissue, necrotic tissue and tumor tissue.

18. The system of claim 15, wherein the optical coherence tomography probe includes an optical fiber and a protective transparent tube, the optical fiber disposed within the protective transparent tube.

19. The system of claim 18, wherein the protective transparent tube includes a nitinol tube with a polytetrafluoroethylene distal end.

20. The system of claim 18, wherein the protective transparent tube comprises polytetrafluoroethylene.

21. The system of claim 15, wherein the biopsy needle is about 5 feet in length.

22. The system of claim 15, wherein the biopsy needle is a 19 gauge needle.

23. An optical coherence tomography probe comprising:
a single-mode optical fiber;
a spacer coupled to the single-mode fiber; and
an optical element coupled to the spacer, wherein the optical element includes a gold coating on a surface of the optical elements.

24. The probe of claim 23, further comprising a protective transparent tube surrounding a segment of the optical coherence tomography probe.

25. The probe of claim 23, wherein a proximal end of the spacer is fused to an end of the single-mode fiber.

26. The probe of claim 23, wherein the optical element comprises at least one of a ball lens or gradient-index fiber.

27. The probe of claim 23, wherein the optical element is fused to a distal end of the spacer.

28. The probe of claim 26, wherein the ball lens is a 45 degree polished ball lens.

29. The probe of claim 28, wherein the gold coating is on a polished surface of the 45 degree polished ball lens.
